(19) United States
(12) Patent Application Publication
(10) Pub. No.: US 2019/0185805 A1
(43) Pub. Date: Jun. 20, 2019

(54) AUTOMATED ELECTROPORATION OF SINGLE CELLS IN MICRO-WELL PLATES

(71) Applicant: INFINITESIMAL LLC, Skokie, IL (US)

(72) Inventors: John Kohoutek, Skokie, IL (US); Vincent Lemaitre, Skokie, IL (US)

(21) Appl. No.: 16/226,029

(22) Filed: Dec. 19, 2018

Related U.S. Application Data
(60) Provisional application No. 62/607,642, filed on Dec. 19, 2017.

(51) Int. Cl. C12M 1/42; C12M 1/32; C12M 1/12; G01N 33/487; G01N 35/10

(52) U.S. Cl. CPC C12M 35/02; C12M 23/12; G01N 2035/1048; G01N 33/48707; G01N 35/10; C12M 25/06

(57) ABSTRACT

Systems and methods are described for operating an electroporation system with single cell resolution. The system controllably adjusts an (x,y) position of a multi-well plate to position a particular well relative to an electrode, a pipette, and a microscope camera that are stationary in the (x,y) plane. The same motorized (x,y) stage is also controlled to position a particular one of a plurality of interchangeable microfluidic probes (e.g., micropipettes) below a microfluidic probe coupler, wherein the coupler is also stationary in the (x,y) plane.

### RELATED APPLICATIONS

This application claims the benefit of U.S. Provisional Patent Application No. 62/607,642, filed Dec. 19, 2017, entitled “AUTOMATED ELECTROPORATION OF SINGLE CELLS IN MICRO-WELL PLATES,” the entire contents of which are incorporated herein by reference.

### STATEMENT REGARDING FEDERALLY SPONSORED RESEARCH OR DEVELOPMENT

This invention was made with government support under grant R44 GM101833 from the National Institutes of Health (NIH). The government has certain rights in the invention.

### BACKGROUND

The present invention relates to systems and methods for performing electroporation/transfection.

### SUMMARY

In various embodiments, the invention provides a) automated imaging and localization of individual cells or clusters of cells (e.g., induced pluripotent stem cells (“iPSCs”)) plated in a multi-well plate (e.g., 96-well plate), b) automated single-cell electroporation, c) a user interface with a wide choice of protocols and low noise electric pulses, d) automated cleaning of the electrode embedded in the micropipette housing, and e) automated changing of microfluidic probes (e.g., up to 8 micropipettes per plate). In some embodiments, the system is an integrated device including a microscope with phase contrast and fluorescence capabilities, a camera linked to the microscope, image analysis software, a high resolution motorized x-y stage, a z-axis piezo stage holding a micropipette with an embedded electrode, and a cell contact recognition algorithm for automated single-cell electroporation. Electronics hardware, control software, and a PC user interface will be used to automate the process. Other features include a micropipette loading bay for automated change of pipette, a camera or laser diodes-sensors assembly for micropipette position calibration, and a waste bin. In a particular embodiment, the system is enclosed within an environmental chamber that allows for temperature and CO₂ control. The system will provide unprecedented levels of efficiency, viability, and ease of use compared to traditional workflows based on bulk electroporation.

Other aspects of the invention will become apparent by consideration of the detailed description and accompanying drawings.

### BRIEF DESCRIPTION OF THE DRAWINGS

FIG. 1 is a perspective view of a system for automated single-cell electroporation using a multi-well plate and multiple transfection micropipettes according to one implementation.

FIG. 2A is a schematic elevation view of the system of FIG. 1.

FIG. 2B is a schematic overhead view of the system of FIG. 1.

FIG. 3 is a block diagram of a control system for operating the system of FIG. 1.

FIG. 4 is a flowchart of a method for performing single-cell electroporation using the system of FIG. 1 and the control system of FIG. 3.

FIG. 5 is a flowchart of a method for analyzing cell colonies using the system of FIG. 1 and the control system of FIG. 3.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

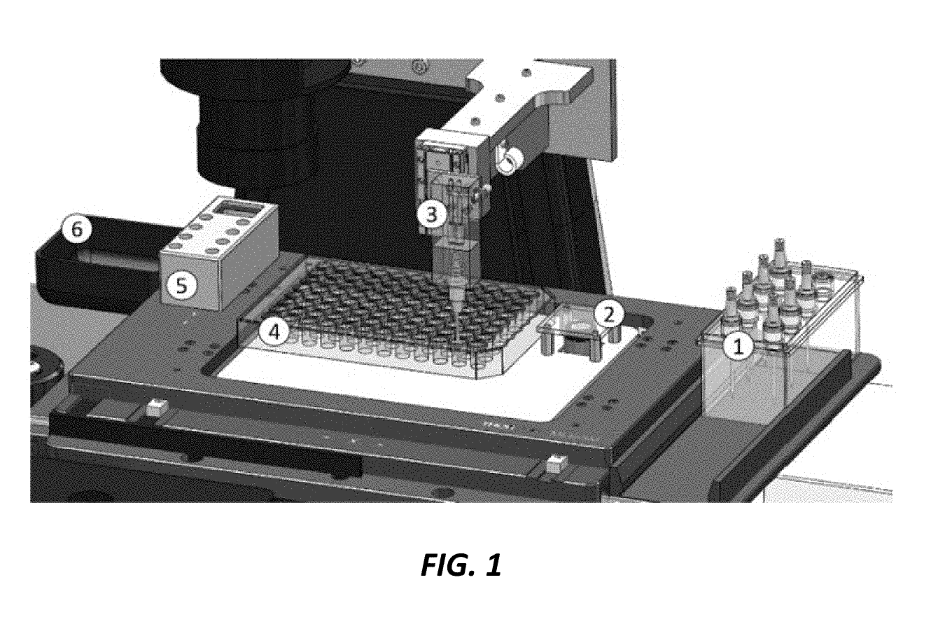

FIG. 1 illustrates a system for performing single-cell electroporation using a multi-well plate and, in at least some situations, multiple micropipettes. The system includes a motorized (x,y) stage and a separate z-stage. A micropipette loading bay (1), a calibration camera (2), an electrode cleaning bay (5), and a waste bin (6) are positioned on (or, in some implementations, affixed to) the motorized (x,y) such that the controlled movement of the motorized (x,y) stage causes movement of these components as well. The motorized (x,y) stage also includes a surface for placement of a multi-well plate (4). In the example of FIG. 1, the multi-well plate is a 96-well plate. The surface of the motorized (x,y) stage underneath where the multi-well plate (4) is positioned is at least partially transparent such that another camera positioned under the motorized (x,y) stage (discussed in further detail below) is able to capture image data of materials held in each well of the multi-well plate (4). For example, the surface for holding the micro-well plate (4) may be constructed of a transparent material such as, for example, glass.

As described in further detail below, an electrode and a micropipette holder are integrated into the z-stage (3) and the z-stage (3) is configured to controllably move the electrode and a micropipette (when attached) in the z direction (i.e., up and/or down). The z-stage (3) is configured in the example of FIG. 1 is separate from the motorized (x,y) stage such that the controlled movement of the motorized (x,y) stage does not cause movement of the z-stage (3). Conversely, movement of the z-stage (3) in the z-direction does not cause movement of the motorized (x,y) stage or movement of any components positioned on the motorized (x,y) stage.

In some implementations, the system of FIG. 1 includes an integrated environment chamber that is configured to regulate environmental conditions such as, for example, temperature and CO₂ levels. In some other implementations, the system of FIG. 1 is configured to be partially or entirely positioned within a separate environment chamber that is not necessarily integrated structurally or functionally into the system of FIG. 1.

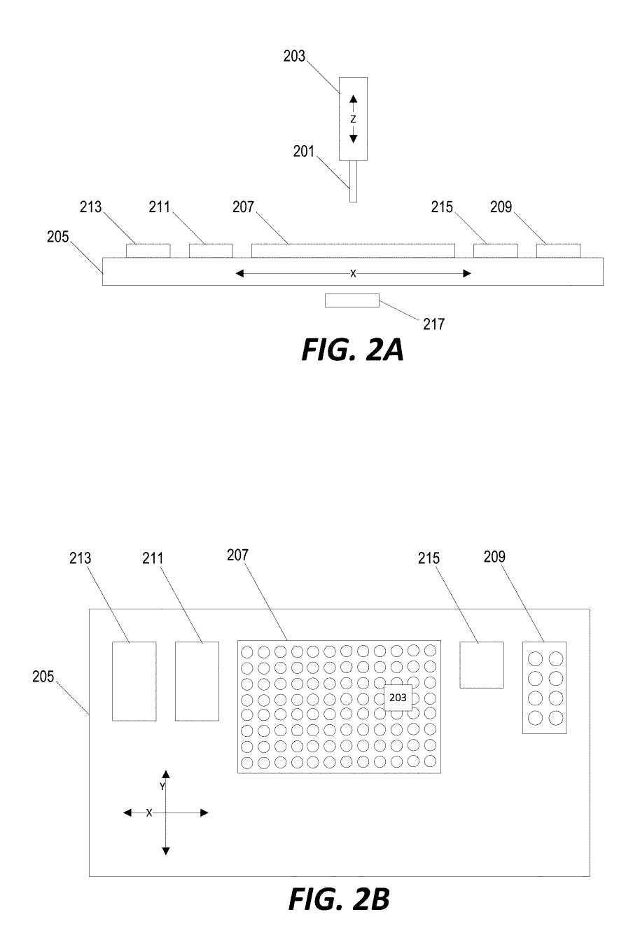

FIGS. 2A and 2B further illustrate certain components of the system of FIG. 1 and their movement capabilities. FIG. 2A shows an electrode 201 coupled to the z-stage 203. Controlled actuation of the z-stage 203 causes the electrode 201 to move upward and/or downward in the z-direction. The motorized (x,y) stage 205 is positioned below the z-stage 203 with the multi-well plate 207, a micropipette loading bay 209, an electrode cleaning bay 211, a waste bin 213, and a calibration camera 215 positioned on (or affixed to) the motorized (x,y) stage 205 such that controllable movement of the motorized (x,y) stage 205 also moves these components in the x- and y-directions.

An inverted microscope lens 217 is positioned directly below the electrode 201 in the x- and y-directions and is located below the motorized (x,y) stage 205. The inverted microscope lens 217 is not coupled to the motorized (x,y) stage 205 and, therefore, does not move in the x- and y-directions. The inverted microscope lens 217 is also not coupled to the z-stage 203 and does not move in the z-direction.

Accordingly, in the configuration illustrated in FIGS. 2A and 2B, only the motorized (x,y) stage 205 and the components positioned on (or affixed to) the motorized (x,y) stage 205 are capable of controlled movement in the x- and y-directions—the z-stage 203, the electrode 201, and the inverted microscope lens 217 remain stationary in the x-direction and in the y-direction. Furthermore, in the configuration illustrated in FIGS. 2A and 2B, only the z-stage 203 and the component positioned on (or affixed to) the z-stage 203 are capable of controlled movement in the z-direction—the motorized (x,y) stage 205 and the inverted microscope camera 217 remain stationary in the z-direction.

In this way, a particular component positioned on or affixed to the motorized (x,y) stage 205 can be positioned underneath the z-stage 203 and above the inverted microscope lens 217 by controllably adjusting the (x,y) position of the motorized (x,y) stage 205. In the example of FIG. 2B, a particular well of the multi-well plate 207 has been controllably positioned underneath the z-stage 203. Once a particular component is controllably positioned below the z-stage 203 and above the inverted microscope camera 217, the z-stage 203 can be operated to lower the electrode 201 (and/or a micropipette) towards the component as described in further detail below.

The example discussed above in reference to FIGS. 1, 2A, and 2B is only one possible implementation of the system. Other implementations may include more components, fewer components, or different configurations of components. For example, in the system of FIGS. 1, 2A, and 2B, the multi-well plate 207, the micropipette loading bay 209, the electrode cleaning bay 211, the waste bin 213, and the calibration camera 215 are all coupled to and moved by the same motorized (x,y) stage 205. However, in some other implementations, multiple motorized (x,y) stages might be utilized to separately control and adjust the position of certain components in the (x,y) plane. For example, a first motorized (x,y) stage might be configured to move the multi-well plate 207 and the calibration camera 215 while a second motorized (x,y) stage is used to adjust the position of the micropipette loading bay 209, the electrode cleaning bay 211, and the waste bin 213.

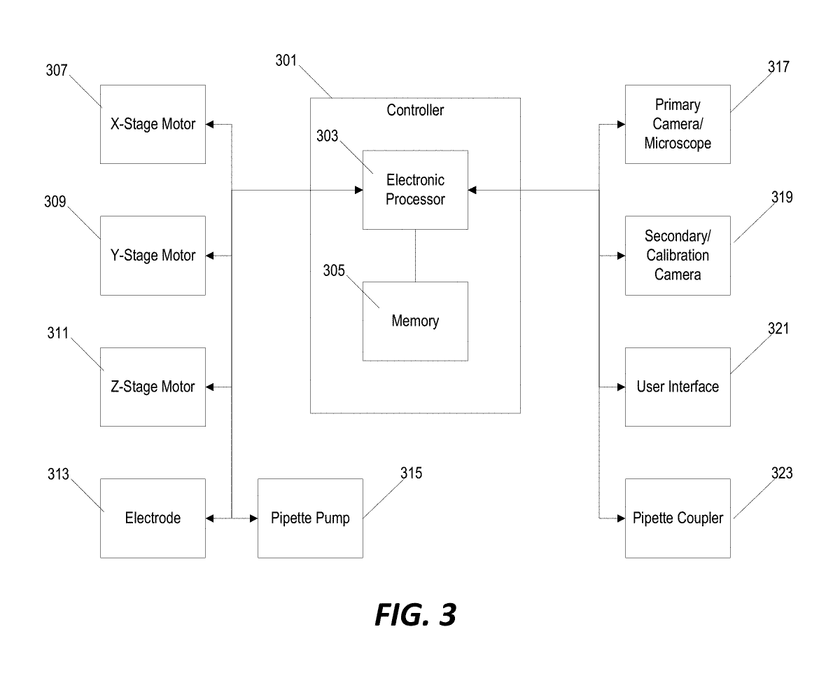

FIG. 3 illustrates an example of a control system for operating the system as described in reference to FIGS. 1, 2A, and 2B. A controller 301 includes an electronic processor 303 and a non-transitory computer-readable memory 305. The memory 305 stores instructions that, when executed by the electronic processor 303, provide the functionality of the controller 301—including, for example, the functionality described herein. The controller 301 is communicative coupled to an x-stage motor 307 and a y-stage motor 309 and transmits control signals/instructions to the x-stage motor 307 and the y-stage motor 309 to controllably adjust the (x,y) position of the motorized (x,y) stage 205. The controller 301 is also communicative coupled to a z-stage motor 311 and is configured to transmit control signals/instruction to the z-stage motor 311 to controllably adjust a height of the z-stage 203.

The controller 301 is also communicatively coupled to an electrode 313 (e.g., electrode 201 in FIGS. 2A and 2B), a pipette pump 315, and a pipette coupler/clamp 316. The controller 301 uses the electrode 313 to detect contact between the pipette and a cell and applies a current to the cell for electroporation. The controller 301 operates the pipette pump 315 to dispense a transfection agent to the cell while performing electroporation. As discussed further below, the controller 301 operates the pipette coupler/clamp 316 to selectively attach and hold a pipette on the z-stage 203 and to release the pipette over the waste bin 213 to dispose of the pipette.

The controller 301 is also communicatively coupled to a primary camera/microscope 317 (e.g., the inverted microscope camera 217 in FIG. 2A), a secondary calibration camera 319, and a user interface 321. As discussed further below, the controller 301 is configured to determine a location of a cell within each well of the multi-well plate based image data received from the primary camera/microscope 317. The controller 301 is configured to calibrate the system for a new pipette by identifying a precise location of the pipette tip in the (x,y) coordinate frame of the motorized (x,y) stage 205 using the output of the secondary/calibration camera 319. Finally, the controller 301 is configured to interact with the user interface 321 to receive system control information (i.e., a user-defined instruction for which pipette held in the micropipette loading bay 209 to use for electroporation of each individual well or rows of wells of the multi-well plate 207). In some implementations, the controller 301 is further configured to display certain image data on the user interface 321 for viewing an analysis by a user.

In some implementations, the user interface 321 can include a display screen and a user input mechanism. For example, a touch-screen interface can be incorporated into the integrated device illustrated in FIG. 1. In other implementations, the user interface 321 may be implemented as a separate computer system (e.g., a desktop or tablet computer) configured to communicate with the controller 301 and to display a graphical user interface. In still other implementations, the controller 301 itself may be implemented as a part of the desktop or table computer system.

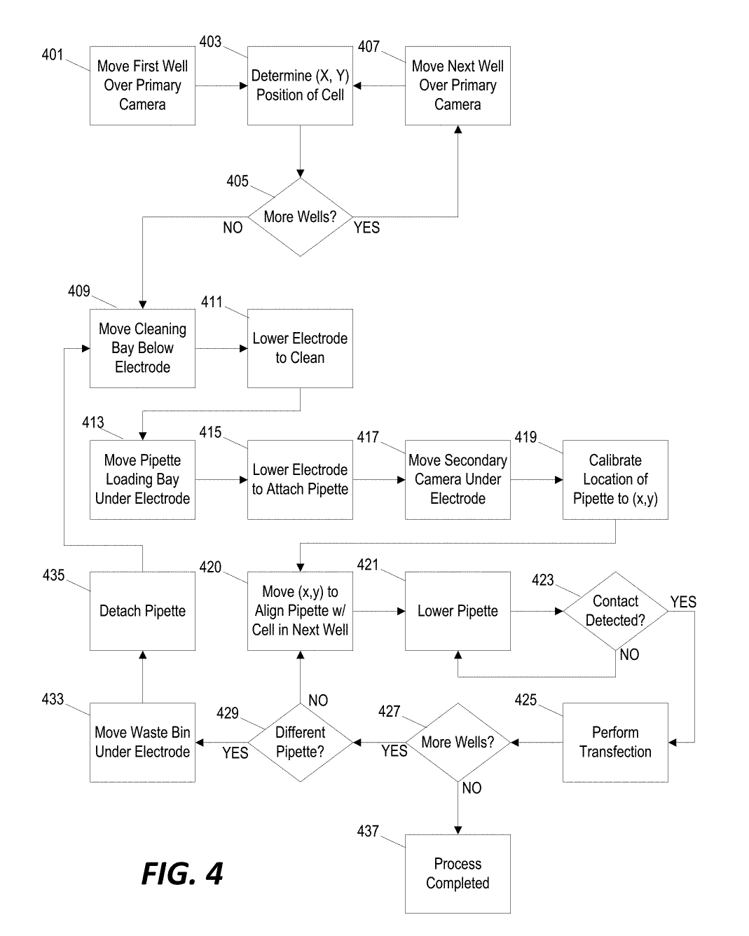

FIG. 4 illustrates a method, implemented by the control system of FIG. 3, for performing automated single-cell electroporation using the system of FIGS. 1, 2A, and 2B. Prior to executing the method of FIG. 4, a user has prepared the multi-well plate by plating one iPSC per well in a matrigel-coated 96-well plate containing an embedded electrode (e.g., Axion Biosystems). This plating can be performed manually using a multichannel pipette and adequate dilution or utilizing an automated system such as, for example, NamoCell (www.namocell.com), On-Chip Bio (www.on-chipbio.com/spis/), or other cell cytometry equipment. Although this specific example discusses iPSCs and a matrigel-coated 96-well plate, other implementations may be configured for other cell types and may utilize other types of multi-well plates (e.g., with different coating or with a different number of micro-wells per plate).

After cell plating, the culture plate is placed on the motorized (x,y) stage 205. The controller 301 operates the x-stage motor 307 and the y-stage motor 309 to position a first well of the multi-well plate 207 above the primary microscope camera 317 (step 401). Based on image data from the primary microscope camera 317, the controller 301 determines an (x,y) position of the cell in the first well (step 403). The controller 301 then adjusts the (x,y) position of the motorized (x,y) stage 205 to position the next well of the multi-well plate 207 over the primary microscope camera 317 (step 407) and determines the (x,y) position of the cell in that well based on image data from the primary microscope camera and machine learning computational algorithms (step 403). This process is repeated for all utilized wells of the multi-well plate 207 (step 405).

After the controller 301 has determined an (x,y) position of the cell in each individual well, the controller 301 operates the motorized (x,y) stage 205 to position the cleaning bay 211 below the electrode 201 (step 409) and then operates the z-stage motor 311 to lower the electrode 201 into the cleaning bay 211 for cleaning (step 411). The electrode 201 is then lifted and the motorized (x,y) stage is moved to position a particular micropipette in the pipette loading bay 209 below the z-stage 203 (step 413). The z-stage 203 then lowers and the micropipette is attached (step 415). The z-stage 203 lifts the attached pipette and the motorized (x,y) stage moves the secondary camera 319 underneath the attached pipette (step 417). Although this example describes cleaning the electrode before a micropipette is attached, in some implementations, the electrode may be incorporated directly into the pipette. Accordingly, in such implementations, a separate washing step before coupling a new pipette may not be necessary as the entire electrode is replaced each time a new pipette is attached. Accordingly, the washing step may be adjusted or the washing step (and the cleaning bay 211) might be omitted entirely in some implementations. Conversely, in some implementations, the controller 301 may be configured to operate the system to clean the electrode between every transfection—and not only before attaching a new/different pipette.

Based on image data from the secondary camera 319, the controller 301 determines a precise location of the pipette tip relative to the (x,y) coordinate frame of the motorized (x,y) stage 205 (step 419). In some implementations, the calibration process implemented by the controller 301 is configured to determine a location of the pipette tip in relation to the cell in the multi-well plate. As described above, the controller 301 has identified a location of the cell in the x,y plane based on the movement coordinates of the motorized (x,y) stage 205 (i.e., what movements of the motorized (x,y) stage 205 are required in order to position the cell above the microscope camera 317). Because lens/objective of the calibration camera 319 is fixed relative to the motorized (x,y) stage 205, the controller 301 is able to determine a location of an individual cell in a well of the multi-well plate relative to the location of the calibration camera 319 in the (x,y) coordinate frame of the motorized (x,y) stage 205. By then moving the motorized (x,y) stage 205 to center the tip of the pipette over the calibration camera 319, the controller 301 is able to determine a location of the pipette tip relative to the (x,y) coordinate frame of the motorized (x,y) stage 205. Because, after this calibration process, the position of the pipette tip and the location of the cell are now both defined relative to the (x,y) coordinate frame of the motorized (x,y) movement stage 205, the controller 301 is able to control the movement of the motorized (x,y) stage 205 to precisely position the cell directly below the pipette tip.

Once the position of the pipette tip is calibrated, the motorized (x,y) stage 205 moves to align the pipette tip with the location of the cell (determined in step 401 above) in the x- and y-directions (step 420). The z-stage motor 311 is then actuated to lower the pipette (step 421) until contact between the pipette tip and the cell is detected (step 423). Once contact is detected (step 42), the system is operated to perform transfection/electroporation on the cell within the first well (step 425). In some implementations, the controller 301 is configured to detect contact between the pipette tip and the cell and/or to perform electroporation using systems and methods such as described in U.S. Provisional Application No. 62/454,399.

After performing transfection, on the cell in the first well of the multi-well plate 207, the controller repeats the process for each additional well (step 427). The controller 301 determines, based on user-defined criteria, whether electroporation of the cell in the next well is to be performed using the same pipette (e.g., the same transfection agent) (step 429). If so, the motorized (x,y) stage 205 is moved to align the pipette tip with the location of the cell in the next well (step 420) and performs electroporation of that cell (i.e., repeating steps 421, 423, and 425). However, if a different pipette is required for electroporation of the cell in the next well, the motorized (x,y) stage 205 moves the waste bin 213 under the electrode (step 433) and the attached micropipette is released into the waste bin 213 (step 435). The electrode is then cleaned (steps 409 and 411) before attaching a new pipette (steps 413 and 415). Each time a new pipette is attached from the pipette loading bay 209, the calibration procedure (steps 417 and 419) is repeated to determine a precise location of the new pipette tip relative to the (x,y) coordinate frame of the motorized (x,y) stage 205.

This process is repeated until electroporation of the cell in each well of the plate has been completed (steps 427). In some implementations, the user can define a subset of wells for transfection (e.g., through a software interface) so that the transfection procedure might be repeated for all wells of the plate or for only the subset of wells as defined by the user. After performing electroporation on the cell in the last well of the multi-well plate, the automated process is completed (step 437). A new multi-well plate can now be placed in on the motorized (x,y) stage 205 (and, in some cases, a new set of micropipettes/transfectants can be placed in the pipette loading bay 209) and the process can be repeated.

In this example, the micropipettes are released into the waste bin 213 before switching to a new micropipette. However, in some configurations, the controller 301 is configured to cause the motorized (x,y) stage to position the pipette loading bay 209 below the micropipette after use and to return the micropipette to its original location in the pipette loading bay 209 before cleaning the electrode and attaching a different micropipette to the z-stage 203.

After several days of culture, the wells will each contain a colony originating from a single transfected cell. The colonies can be dissociated (e.g., using EDTA) and then transferred into new culture wells or tubes for expansion or screen, such as DNA sequencing and qPCR analysis. This procedure can be performed with a multichannel pipette, or with automated liquid handling systems available, for example, from Eppendorf, ThermoFisher, or Hamilton, among others.

The system illustrated in FIGS. 1 through 3 can also be used for post-transfection analysis using the primary microscope camera 317, which, in some configurations, includes phase-contrast and fluorescence video-microscopy capabilities. The user will be able to follow cell growth and morphology by placing the plate back in the system where pictures of every colony will be taken automatically, with the motorized (x,y) stage 205 moving rapidly from well-to-well.

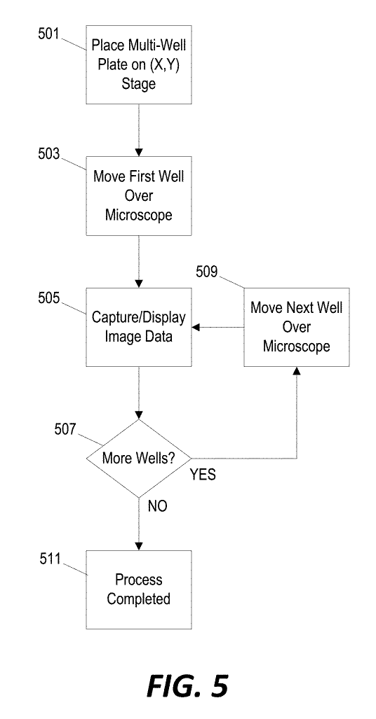

FIG. 5 illustrates one example of an automated process for capturing post-transfection image data for monitoring and analysis. The multi-well plate 207 is placed back in its position on the motorized (x,y) stage 205 (step 501). The controller 301 then controls the x-stage motor 307 and the y-stage motor 309 to move the first well over the primary microscope camera 317 (step 503). Image data is then captured by the primary microscope camera 317 and stored to memory 305 (step 505). The system then moves the next well over the primary microscope camera (step 509) and captures image data for the next well (step 505). The process of moving the multi-well plate (step 509) and capturing image data (step 505) is repeated until image data is captured for every well of the multi-well plate 205 (step 507) at which time the process is completed (step 511).

In various implementations, systems and methods similar to those described above may be utilized to automatically and serially bring each well over the lens of a microscope for image analysis. The (x,y) position of each cell will be located and recorded using features of the imaging software. The system will then clean the electrode embedded in the micropipette housing and attach a glass micropipette, containing the desired transfectant, to the micropipette holder on the z-axis piezo stage. Multiple micropipettes, each containing a specific transfectant, can be used and replaced during the transfection of rows in, for example, a 96-well plate. Once attached to the holder, the precise (x,y) position of the micropipette tip will be calibrated using a secondary camera.

The motorized stage holding the culture plate will then serially and precisely move each well/cell under the vertically-positioned glass micropipette containing the transfectant and the embedded platinum electrode. The micropipette will move down using a piezo stage and transfection will be performed automatically, using a cell contact algorithm. In the specific examples described above, transfection speeds of 10 cells/min can be achieved, based on the distance needed to travel between each well and the maximum speed of the x,y and z stages, and accounting for a final slower z-approach. Therefore, one 96-well plate will be processed in less than 15 minutes outside the culture incubator, which will not affect iPSCs based on our experience with these cells. However, in some implementations, the actual transfection speeds for an individual cell and for an entire microwell plate may be slower or faster. In a particular embodiment, a microscope heated stage will be added to maintain the temperature of the media at 37° C. In other embodiments, some or all of the system illustrated in FIG. 1 may be contained in an environmental chamber capable of controlling temperature and CO₂ as discussed above. In such embodiments, the system is configured such that a multi-well plate held by the motorized platform remains positioned within the environmental chamber during the operation of the system as described above.

The pulse parameters (pulse type, voltage, time, and frequency), the wells to transfect and the micropipettes to be used, will be determined by the user via a PC software interface. After transfection, the plate will be placed back into the culture incubator for cell growth.

In some implementations, a Nikon Eclipse Ti inverted microscope and CoolSNAP HQ2 CCD camera or equivalent will be used. The stage will be programmed to move to specific locations within the system, e.g., micropipette loading bay, as well as automated to move to the specific (x,y) transfection point for each cell in a 96-well plate. To control the positioning of the (x,y) stage, a C++ wrapper will be written to communicate with the Application Program Interface (API) of the (x,y) stage controller. An example of commercially available (x,y) stage API's include the PI MikroMove software (Physik Instrumente Corp.).

In some implementations, the cell location within each well of the 96-well plate are determined using an image recognition algorithm where the well is moved over the microscope camera and scanned over the area around the well center in low magnification. The images captured from the microscope camera are processed in real-time using methods similar to those frequently employed in facial detection and recognition such as a cascade of classifiers on image features which has been previously trained on images of cells. Additional detection methods will be employed in conjunction for error checking based on statistical methods—such as Principal Component Analysis (PCA) as applied in Eigenfaces—or mathematical means such as gradient analysis and thresholding. These methods have been proven for detection of inanimate and living targets.

The output from this detection algorithm will allow the recording of cell locations for each well of the 96-well plate. To automatically transfect cells in each well, an algorithm for automatic calibration interfaces the positioning of the (x,y) stage, the microscope camera feed and a secondary camera feed. In some implementations, the culture plate is fixed during the testing process. In addition to locating the cell within the microwell, the controller is configured to use data from the microscope camera to resolve the boundary of the microwell and determine the well center point. The secondary camera will be fixed onto the (x,y) stage near the microwell plate. This camera, whose position is known in the (x,y) stage frame of reference, will be used to calibrate the minutely varying location of the micropipette tip by utilizing a similar image recognition algorithm as described for determining the cell position above. It will then measure the micropipette tip location and stores it as an offset from the secondary camera's image center. Thus, with this secondary camera and the microscope camera calibrated, each time a microplate well and micropipette will be installed, their locations will be calibrated in a repeatable manner in the (x,y) stage coordinate frame.

Using the (x,y) offsets given by the algorithms above, the controller then uses a stage motion library to collocate the cell with the tip of the micropipette in (x,y) in the transfection area. The micropipette will only move up or down in the z direction, as it will be mounted on a single linear stage with that orientation. In some implementations, the controller applies an automatic cell contact algorithm that uses an electrical resistance measurement to detect when the small opening of the micropipette has come into contact with the cell membrane. Using this algorithm and the collocated (x,y) position given by the algorithms above, we will precisely contact the individual cells in each well and transfect them using electroporation voltages controlled by electronics and software.

In some implementations, some or all of the control functionality may be implemented using software operating on a desktop computer system to initiate/terminate the automated methods, to define the transfection criteria (e.g., which micropipettes to use for which wells), etc. The software may be configured to contain a communications library to talk to a microcontroller unit 301 on a custom printed circuit board. This microcontroller will control two important chips, an analog to digital converter and a digital to analog converter. The analog to digital converter will read low-voltage pulses sent to the cell sample. These pulses will decay due to a series capacitor, allowing us to measure the resistance through the decay rate of the pulse. The digital to analog converter will allow us to translate arbitrary waveforms with a limited number of points into repeated pulses that we can send to the sample for transfection. These pulses are amplified by a high voltage amplifier—thus the pulse shape, magnitude, and frequency are all customizable by the user in the software. The software can also implement the algorithms which encompasses the auto-z, cell recognition, micropipette tip recognition, and calibration routines. These routines use proven image recognition algorithms to determine where the microwell, cell and micropipette tip are relative to each other in terms of the (x,y) stage coordinate frame. The calibration and stage positioning systems will be controlled by the output of these algorithms as well as the user preferences for what gets transfected where. It will have a window in which the user can visualize the 96-well plate, color coded for each type of cell in the plate. Some implementations may also include a point-and-click type feature, where the user can select from the available transfectant and then click in the visualization of their 96-well plates to tell the system which transfectant they would like in which cell.

Furthermore, although the examples described above describe situations in which only a single cell is located in each well of a multi-well plate, in some implementations, the systems and methods can be adapted for uses where multiple cells are present in each well. For example, a well in a multi-well plate may include a cluster of cells and the systems described above can be configured/used to perform transfection in one or more individual cells in the same well. In one particular example, each well of the multi-well plate contains a cluster of cells and the system is configured/used to transfect one cell in each well with a plasmid containing an antibiotic resistant gene. Using antibiotic selection, one can then generate cell lines in each well.

Lastly, although the examples presented above describe using a secondary camera to determine a position of the tip of the micropipette or microfluidic probe, in some implementations, other techniques and/or mechanisms are used. For example, in some implementations, a laser diode/sensor assembly is included instead of the camera and configured to determine a position of the tip of the micropipette/microfluidic probe. In various implementations, the laser diode/sensor assembly can be coupled to the motorized platform (such that movement of the motorized platform causes movement of the assembly) or can be coupled to another component of the system (such that movement of the motorized platform does not cause movement of the assembly).

Thus, the invention provides, among other things, a system and method for automated electroporation of single cells in micro-well plates with interchangeable microfluidic probes (e.g., micropipettes). Various features and advantages of the invention are set forth in the following claims.

What is claimed is:

1. An electroporation system comprising:
a motorized platform configured to hold a multi-well plate, wherein a position of the motorized platform is controllably adjustable in an x-direction and in a y-direction;
a camera, wherein movement of the motorized platform does not change a position of the camera in the x-direction or in the y-direction;
a motorized z-stage configured to controllably adjust a position of a microfluidic probe in a z-direction, wherein movement of the motorized platform does not change a position of the motorized z-stage in the x-direction or in the y-direction; and
a controller configured to:
adjust a position of the motorized platform in the x-direction and in the y-direction to position a well of the multi-well plate in a field of view of the camera, wherein the field of view of the camera is aligned with the microfluidic probe,
determine a position of a cell in the well of the multi-well plate in an (x, y) coordinate frame based on a controlled position of the motorized platform and a detected location of the cell in the first well in an image captured by the camera,
adjust a position of the motorized platform to align the determined position of the cell in the (x, y) coordinate frame with a position of the microfluidic probe in the (x, y) coordinate frame, and
adjust a position of the motorized z-stage in the z-direction to cause a tip of the microfluidic probe to contact the cell.

2. The electroporation system of claim 1, wherein the camera is positioned on a side of the motorized platform opposite the motorized z-stage, and wherein the motorized platform includes a transparent section positionable in the field of view of the camera such that each one or more wells of the multi-well plate can be imaged by the camera through the motorized platform.

3. The electroporation system of claim 1, further comprising a second camera fixedly coupled to the motorized platform, wherein the controller is further configured to determine the position of the microfluidic probe in the (x, y) coordinate frame by
adjusting a position of the motorized platform in the x-direction and in the y-direction to position the tip of the microfluidic probe in a field of view of the second camera,
identifying a location of the tip of the microfluidic probe in an image captured by the second camera while positioned with the tip of the microfluidic probe in the field of view of the second camera, and
determining the position of the tip of the microfluidic probe in the (x, y) coordinate frame based on a controlled position of the motorized platform when the image is captured by the second camera and the identified location of the tip of the microfluidic probe in the image captured by the second camera.

4. The electroporation system of claim 3, wherein the microfluidic probe includes a micropipette, and further comprising a micropipette loading bay fixedly coupled to the motorized platform, wherein the controller is further configured to
adjust a position of the motorized platform in the x-direction and in the y-direction to align a micropipette held in the micropipette loading bay with the motorized z-stage in the (x, y) coordinate frame, and
adjust a position of the motorized z-stage in the z-direction to couple the micropipette to the motorized z-stage, and
wherein the control is configured to determine the position of the micropipette in the (x, y) coordinate frame after the micropipette from the micropipette loading bay is coupled to the motorized z-stage.

5. The electroporation system of claim 4, further comprising a waste bin coupled to the motorized platform, wherein the controller is further configured to discard a micropipette after use by
adjusting a position of the motorized platform in the x-direction and in the y-direction to align the waste bin with the micropipette in the (x, y) coordinate frame, and
releasing the micropipette from the motorized z-stage.

6. The electroporation system of claim 4, further comprising:
a cleaning bay fixedly coupled to the motorized platform; and
an electrode fixedly coupled to the motorized z-stage, wherein the electrode is positioned inside the micropipette when the micropipette is coupled to the motorized z-stage,
wherein the controller is further configured to wash the electrode before coupling the micropipette to the motorized z-stage by
adjusting a position of the motorized platform in the x-direction and in the y-direction to position the cleaning bay below the electrode, and
adjusting a position of the motorized z-stage in the z-direction to lower the electrode into a cleaning medium held in the cleaning bay.

7. The electroporation system of claim 1, further comprising a cleaning bay fixedly coupled to the motorized platform, wherein the controller is further configured to
adjust a position of the motorized platform in the x-direction and in the y-direction to position the cleaning bay below the tip of the microfluidic probe, and
adjust a position of the motorized z-stage in the z-direction to lower the tip of the microfluidic probe into a cleaning medium held in the cleaning bay.

8. The electroporation system of claim 1, wherein the microfluidic probe includes a micropipette, and further comprising a pipette pump coupled to the motorized z-stage and configured to regulate suction of the micropipette, wherein the controller is further configured to
detect contact between the tip of the micropipette and the cell in the well of the multi-well plate, and
perform transfection of the cell by regulating a voltage or a current of an electrode positioned inside the micropipette and operating the pipette pump to expel a fluid media from the micropipette.

9. The electroporation system of claim 1, wherein the controller is further configured to
determine a position of a plurality of cells, each cell of the plurality of cells positioned in a different well of a plurality of wells of the multi-well plate, by
repeatedly adjusting a position of the motorized platform to iteratively position each well of the plurality of different wells in the field of view of the camera and
determining the position of each cell in the (x, y) coordinate frame based on the controlled position of the motorized platform and a detected location of each cell in images captured by the camera, and
perform transfection of each cell of the plurality of cells by
repeatedly adjusting a position of the motorized platform to iteratively align each cell of the plurality of cells with a position of the microfluidic probe in the (x, y) coordinate frame and
repeatedly adjusting a position of the motorized z-stage in the z-direction to iteratively cause the tip of the microfluidic probe to contact each cell of the plurality of cells.

10. The electroporation system of claim 1, the controller is configured to determine the position of the cell in the well of the multi-well plate by analyzing the image captured by the camera using a machine-learning image processing algorithm.

11. The electroporation system of claim 1, further comprising an environmental chamber configured to regulate temperature and CO₂ within the environmental chamber, and wherein the electroporation system is configured such that the multi-well plate held by the motorized platform is positioned within the environmental chamber.

12. A method of operating an electroporation system, the electroporation system including
a motorized platform configured to hold a multi-well plate, wherein a position of the motorized platform is controllably adjustable in an x-direction and in a y-direction,
a camera, wherein movement of the motorized platform does not change a position of the camera in the x-direction or in the y-direction, and
a motorized z-stage configured to controllably adjust a position of a microfluidic probe in a z-direction, wherein movement of the motorized platform does not change a position of the motorized z-stage in the x-direction or in the y-direction
the method comprising:
adjusting a position of the motorized platform in the x-direction and in the y-direction to position a well of the multi-well plate in a field of view of the camera,
determining a position of a cell in the well of the multi-well plate in an (x, y) coordinate frame based on a controlled position of the motorized platform and a detected location of the cell in the first well in an image captured by the camera,
adjusting a position of the motorized platform to align the determined position of the cell in the (x, y) coordinate frame with a position of the microfluidic probe in the (x, y) coordinate frame, and
adjusting a position of the motorized z-stage in the z-direction to cause a tip of the microfluidic probe to contact the cell.

13. The method of claim 12, further comprising determining the position of the microfluidic probe in the (x, y) coordinate frame by
adjusting a position of the motorized platform in the x-direction and in the y-direction to position the tip of the microfluidic probe in a field of view of a second camera fixedly coupled to the motorized platform,
identifying a location of the tip of the microfluidic probe in an image captured by the second camera while positioned with the tip of the microfluidic probe in the field of view of the second camera, and
determining the position of the tip of the microfluidic probe in the (x, y) coordinate frame based on a controlled position of the motorized platform when the image is captured by the second camera and the identified location of the tip of the microfluidic probe in the image captured by the second camera.

14. The method of claim 13, wherein the microfluidic probe includes a micropipette, and further comprising:
adjusting a position of the motorized platform in the x-direction and in the y-direction to align a micropipette held in a micropipette loading bay with the motorized z-stage in the (x, y) coordinate frame, wherein the micropipette loading bay is fixedly coupled to the motorized platform, and
adjusting a position of the motorized z-stage in the z-direction to couple the micropipette to the motorized z-stage,
wherein determining the position of the micropipette in the (x, y) coordinate frame includes determining the position of the micropipette in the (x, y) coordinate frame after the micropipette from the micropipette loading bay is coupled to the motorized z-stage.

15. The method of claim 14, further comprising discarding a micropipette after use by
adjusting a position of the motorized platform in the x-direction and in the y-direction to align a waste bin with the micropipette in the (x, y) coordinate frame, wherein the waste bin is coupled to the motorized platform, and
releasing the micropipette from the motorized z-stage.

16. The method of claim 14, further comprising washing an electrode fixedly coupled to the motorized z-stage by
adjusting a position of the motorized platform in the x-direction and in the y-direction to position a cleaning bay below the electrode, wherein the cleaning bay is coupled to the motorized platform, and
adjusting a position of the motorized z-stage in the z-direction to lower the electrode into a cleaning medium held in the cleaning bay,
wherein adjusting the position of the motorized z-stage in the z-direction to couple the micropipette to the motorized z-stage includes coupling the micropipette to the motorized z-stage with the electrode positioned inside the micropipette.

17. The method of claim 12, further comprising:
adjusting a position of the motorized platform in the x-direction and in the y-direction to position a cleaning bay below the tip of the microfluidic probe, wherein the cleaning bay is coupled to the motorized platform, and
adjust a position of the motorized z-stage in the z-direction to lower the tip of the microfluidic probe into a cleaning medium held in the cleaning bay.

18. The method of claim 12, wherein the microfluidic probe includes a micropipette, and further comprising:
detecting contact between the tip of the micropipette and the cell in the well of the multi-well plate, and
performing transfection of the cell by regulating a voltage or a current of an electrode positioned inside the micropipette and operating a pipette pump to expel a fluid media from the micropipette.

19. The method of claim 12, further comprising:
determining a position of a plurality of cells, each cell of the plurality of cells positioned in a different well of a plurality of wells of the multi-well plate, by
repeatedly adjusting a position of the motorized platform to iteratively position each well of the plurality of different wells in the field of view of the camera and
determining the position of each cell in the (x, y) coordinate frame based on the controlled position of the motorized platform and a detected location of each cell in images captured by the camera; and
performing transfection of each cell of the plurality of cells by
repeatedly adjusting a position of the motorized platform to iteratively align each cell of the plurality of cells with a position of the microfluidic probe in the (x, y) coordinate frame and
repeatedly adjusting a position of the motorized z-stage in the z-direction to iteratively cause the tip of the microfluidic probe to contact each cell of the plurality of cells.

20. The method of claim 12, wherein at least one well of the multi-well plate includes a plurality of cells, and wherein determining the position of the cell in the well of the multi-well plate includes determining a position of one particular cell of the plurality of cells in a single well of the multi-well plate.
